(19) United States
(12) Patent Application Publication
(10) Pub. No.: US 2020/0029930 A1
(43) Pub. Date: Jan. 30, 2020

(54) ELECTRONIC SYSTEM FOR FOETAL MONITORING

(71) Applicant: NATEO HEALTHCARE, TOULOUSE (FR)

(72) Inventors: THOMAS LANDMAN, TOULOUSE (FR); OLIVIER BEAUDOIN, TOULOUSE (FR)

(21) Appl. No.: 16/337,393

(22) PCT Filed: Oct. 3, 2017
(86) PCT No.: PCT/FR2017/052712

§ 371 Date: Mar. 27, 2019

(30) Foreign Application Priority Data
Oct. 3, 2016 (FR) 16 59535

(51) Int. Cl. A61B 8/08; A61B 8/02; A61B 8/00

(52) U.S. Cl. CPC A61B 8/0866; A61B 8/02; A61B 8/0883; A61B 2562/04; A61B 8/5223; A61B 8/4227; A61B 2503/02; A61B 8/4477

(57) ABSTRACT

An electronic system for foetal surveillance includes at least two ultrasound sensors to emit and receive ultrasound waves either simultaneously, or simultaneously by groups, or sequentially. The ultrasound sensors configured to be positioned on at least one maternal abdominal portion to cover the foetal cardiac structure in movement and carry out measurements of foetal cardiac frequency. The distance between two ultrasound sensors is maintained by a link. A controller controls the ultrasound sensors with a piece of information to control the ultrasound waves sent to at least one ultrasound sensor. A processor to process the ultrasound signals to continuously estimate the position of the heart, the foetal cardiac rhythm and the foetal movements of one or more foetuses on the basis of the signal or signals received from the ultrasound sensors.

### TECHNICAL FIELD OF THE INVENTION

The present invention relates to an electronic system for foetal surveillance. It applies, in particular, to the monitoring either antepartum of a pregnancy or per-partum during delivery.

### PRIOR ART

Tococardiography systems, hereinafter called CTG (initials of CardioTocoGraphy), comprise several types of sensors measuring a plurality of maternal or foetal biophysical diagnostic parameters. The non-invasive external sensors are of two types:
the tocodynamometer which measures uterine activity and contractions,
the ultrasound Doppler sensor which allows to measure the foetal cardiac rhythm.

The ultrasound sensor is placed on the maternal abdomen, and its position must be optimised by the health professional. An elastic belt surrounding the maternal abdomen allows the retention of the sensor at a fixed position and allows to guarantee good coupling.

Certain documents of the prior art also propose tococardiography systems. For example the publication document US2016120500 which allows to measure the foetal cardiac frequency comprising an ultrasound sensor designed to carry out measurements of foetal cardiac frequency when it is fastened to the abdomen of a pregnant woman is known.

However, poor positioning of the ultrasound sensor leads to false measurements and consequently poor diagnoses.

Other documents are also known, such as the document WO2015/082987, US2012/179046 and US2013/123636. However, these documents do not optimise the positioning or the processing of the ultrasound sensors.

If the positioning or if the angulation of the ultrasound sensor does not observe precise rules, risks of poor measurements can occur. In the CTGs of the prior art, the optimisation of the positioning and of the orientation of the Doppler ultrasound sensor is first carried out roughly by a Leopold's Manoeuvre in order to locate the foetal back for example.

Leopold's Manoeuvres are four conventional manoeuvres used to determine the position of the foetus in the uterus.
Leopold A: One or two hands are placed on the top of the uterus and the foetal part felt is identified.
Leopold B: The surface of the palm of one hand is used to locate the back of the foetus while the other hand feels the irregularities, like the hands and the feet.
Leopold C: The thumb and third finger are used to grasp and determine the foetal part presented at the pubic symphysis.
Leopold D: Both hands are used to describe the foetal head.

Then, in the optimisation of the positioning and of the orientation of the ultrasound sensor, the listening to the Doppler sound emitted by the audio monitor of the CTG is used in a fine manner. The audio Doppler rendition must be as powerful and clear as possible.

Poor positioning of the Doppler sensor can have various negative consequences. This can first lead to confusion of the foetal rhythm with the maternal cardiac rhythm and an underestimation of the FCR (initials of Foetal Cardiac Rhythm). The movement of the foetus or the movement of the mother can also require repositioning of the sensor and of the elastic belt. Intervention of the health personnel is then necessary. This can generate organisational problems, when several patients are monitored simultaneously at different locations. It should be added that the optimisation of the positioning and of the orientation of the ultrasound sensor is a procedure requiring the action of personnel with substantial training.

### OBJECT OF THE INVENTION

The present invention aims to overcome these disadvantages.

For this purpose, according to a first aspect, the present invention is aimed at an electronic system for foetal surveillance, remarkable in that it comprises:
at least four ultrasound sensors emitting and receiving ultrasound waves either simultaneously, or simultaneously by groups, or sequentially, the ultrasound sensors being provided in order to be positioned on at least one maternal abdominal portion in order to cover the foetal cardiac structure in movement and carry out measurements of foetal cardiac frequency, the distance between two ultrasound sensors is maintained by a link,
a module for control of the ultrasound sensors comprising a piece of information for control of the ultrasound waves sent to at least one ultrasound sensor,
a module for processing of the ultrasound signals in order to estimate the foetal cardiac rhythm and the foetal movements on the basis of the signal or signals received from the ultrasound sensors.

Via these arrangements, the use of a plurality of ultrasound sensors allows to improve the measurements. Each ultrasound sensor comprises a transducer. The best transducer for the measurement of FCR is that which creates an ultrasound beam in the axis of the transducer that is the most aligned with the movement of the cardiac cavities and which intersects as perpendicularly as possible the mobile interfaces of the foetal cardiac structures. Moreover, the distance between the surface of this optimal sensor and the foetal heart must be as short as possible, in such a way as to minimise the attenuation of the ultrasound beam in the tissue and guarantee the best quality of the Doppler signal.

Via these arrangements, the use of a processing module comprising a piece of information for control of the ultrasound waves allows the use of various non-limiting operating modes such as:
an ultrasound mode for evaluation of the volume of amniotic fluid;
a mode for detection of the foetal cardiac rhythm;
a mode for detection of the foetal movements.

Via these arrangements, the system does not require manual optimisation of the placement of the ultrasound sensors. The system automatically determines the positioning of the foetal heart and modifies its sequence of firing according to the characteristics of the ultrasound signals received.

Advantageously, the system allows to probe, in depth, maternal and foetal tissue that is adjacent to the foetal heart, according to the ultrasound beams generated in the maternal abdomen. These probings allow the estimation of dimensions of structures of interest for diagnosis such as the dimensions of the amniotic cavity.

Another advantage is to allow to monitor, over the time of the examination, the position of the foetal heart or of other foetal organs. During peripartum monitoring, an estimation of the movement of the foetus is possible in order to monitor the progress of the descent of the child in the maternal pelvis. For antepartum, an estimation of the foetal movements (of the foetal body, of the lower members, or of the foetal upper members) is carried out by the module for processing of the ultrasound signals. It is known, since the publication [Manning F A, Platt L D, Sipos L., Antepartum fetal evaluation: development of a fetal biophysical profile score, Am J Obstet Gynecol. 1980 Mar 15;136(6):787-95], that a quantification of these movements allows to verify the well-being of the foetus.

A substantial advantage of the automation of the detection of the foetal heart as well as of the presence of a population of sensors is to allow to reduce or even eliminate the repositionings of the belt and of the ultrasound sensor during the monitoring. If the foetus moves and regardless of its position, the sensors do not have to be moved. The effects are to reduce the risks of poor measurements and of error, to reduce the intervention time of the operator. Another effect is to allow to entrust the positioning of the diagnostic device to personnel with less training, or to the pregnant woman directly.

The invention is advantageously implemented according to the embodiments and the alternatives disclosed below, which are to be considered individually or according to any technically effective combination.

In one embodiment, the number of ultrasound sensors is between 4 and 64, preferably between 24 and 32.

Via these arrangements, the ultrasound sensors form a network.

In one embodiment, the ultrasound sensors together form a triangular, square, rectangular or circular network.

Via these arrangements, the sensors cover a majority of the cases in order to allow to detect the foetal cardiac rhythm well.

In one embodiment, the length of the link is between 30 and 60 mm, preferably between 37 and 45 mm.

In one embodiment, the link is semi-rigid or semi-elastic.

The semi-rigid or semi-elastic link comprises a certain elasticity that allows the torsion and the bending of the material of the link. Thus, movements are possible while leaving a limited range of movement in order to preserve the geometric rigidity of the assembly. The distance between two sensors always remains the same regardless of the position of the belt on the abdomen of the pregnant woman, the volume investigated by all the sensors is thus as large as possible. Thus, the ultrasound beams created by the transducers do not overlap. The ultrasound beams created individually are independent and separate.

In one embodiment, the piece of control information is a signal, one of these characteristics of which is predetermined by at least one of the elements chosen from: the phase, the energy, the amplitude, the frequency and the waveform.

In one embodiment, the piece of control information is independent from one ultrasound sensor to the other and in real time.

In one embodiment, the processing module is connected by a communication element of said system, the communication element being wired or wireless.

In one embodiment, the wireless link uses at least one of the following modes: radio waves, for example UHF (initials of Ultra High Frequency), light waves, for example infrared, sound waves, for example infrasound or ultrasound and/or specifications for communication over a network, for example Bluetooth® (registered trademark), Wi-Fi® (registered trademark) or ZigBee® (registered trademark).

In one embodiment, the system comprises a communicating terminal configured to read or process the data of the system.

In one embodiment, the system comprises one of the elements chosen from: a tocodynamometer, a sensor of pulsed oxygen saturation (SpO2), an electrocardiogram, a thermometer, at least one microphone, an accelerometer, or an electromyogram. The tocodynamometer is the non-invasive instrument that allows to evaluate the forces of the uterine contractions during labour.

### BRIEF DESCRIPTION OF THE DRAWINGS

Other advantages, goals and features of the present invention emerge from the description that is made, for explanatory and in no way limiting purposes, regarding the appended drawings, in which:

FIG. 1 shows a diagram of application of a plurality of ultrasound sensors onto a maternal abdomen according to a specific embodiment of the system forming the object of the present invention;

FIGS. 2 to 7 show various forms of link between ultrasound sensors;

FIG. 8 shows a realisation of a triangular topology of a network of ultrasound sensors; and

FIG. 9 shows a realisation of an architecture of the system.

### DESCRIPTION OF EXAMPLES OF REALISATION OF THE INVENTION

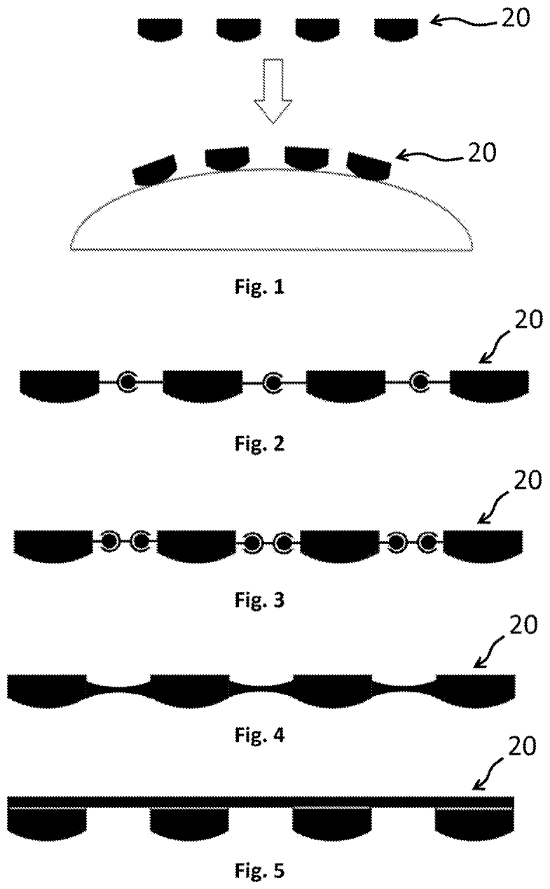

FIG. 1 shows four ultrasound sensors 20 provided in order to be positioned on at least one maternal abdominal portion in order to cover the foetal cardiac structure in movement.

FIGS. 2 to 7 show various forms of link between ultrasound sensors 20. The link between each ultrasound sensor allows to create a belt of ultrasound sensors.

A specific system for retention of a multiplicity of sensors is necessary in order to preserve the geometric distribution of the sensors with respect to each other and also to ensure that all of the sensors form an adaptable assembly adjustable to the morphology of the patient. In order to ensure a reliable and high-performance measurement throughout the examination, it is necessary to ensure good acoustic coupling between each sensor and the skin of the patient (a specific-ultrasounds sequence maybe available in order to verify the quality of this coupling).

In an example of realisation, this coupling is reinforced using a gel, a paste or a cream ensuring good transmission of the ultrasounds. The coupling must also not be disturbed by the movements of the patient or by the contractions. The retention system allows the easy repositioning of the assembly if necessary.

The distance between two ultrasound sensors is maintained by a link.

The link keeps, regardless of the curvature and of the surface on which it is positioned, the geometric distribution of the ultrasound sensors or network.

Six links are presented in FIGS. 2 to 7 that allow to ensure all of the necessary functionalities either via semi-rigid links between the ultrasound sensors (FIGS. 2 and 3) or via semi-elastic links (FIGS. 4 to 7).

In FIGS. 2 and 3, each ultrasound sensor is connected to its closest neighbours via a mechanical interconnection in the form of a ball joint. The articulation is physically on the two linked ultrasound sensors or on the arm connecting them to one another. In one alternative, these ball joints have one degree of rotation in the planes perpendicular to the contact surface of the sensors. In another alternative, these ball joints have two degrees of rotation in the planes perpendicular to the contact surface of the sensors.

As for the degree of rotation that is parallel to the surface, it is blocked in order to keep the geometric rigidity of the sensor distribution.

FIG. 4 uses in order to connect each ultrasound sensor to its closest neighbours a semi-elastic link with a material of the silicone, polyurethane or elastomer type. The rigidity, the elasticity, the torsion and the bending of the material is adapted in order for movements to be possible but with limited ranges of motion in order to preserve the geometric rigidity of the assembly.

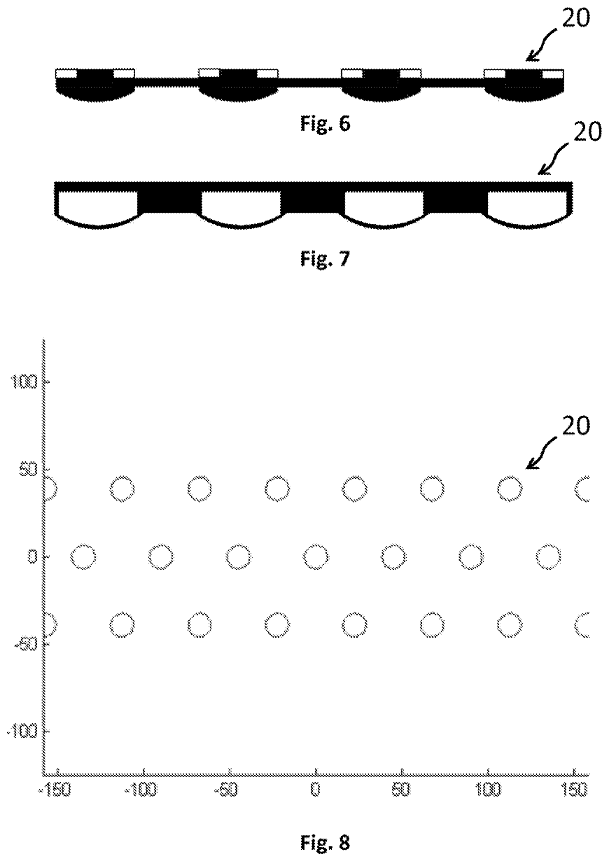

In FIGS. 5 and 6, a substrate of the silicone, polyurethane or elastomer material type is used in order to maintain the geometric rigidity of the assembly and the flexibility necessary in order for the sensors to be able to conform to the contact surface.

In FIG. 5, the ultrasound sensors are assembled by gluing or mechanical fastening onto the surface of the substrate.

In FIG. 6, each ultrasound sensor is positioned in such a way as to pass through openings made in the substrate.

In FIG. 7, after having mechanically positioned the ultrasound sensors with respect to each other, the assembly of the ultrasound sensors is encapsulated by moulding, injection and gluing in a substrate of the silicone, polyurethane or elastomer material type. The substrate in this configuration has acoustic properties favouring the propagation of the ultrasounds as well as properties of biocompatibility because it is the material interfacing with the patient.

FIG. 8 shows a realisation of a triangular topology of a network of ultrasound sensors 20.

In this example, the network is equilateral triangular. A main direction is the lateral-medial direction (LM) (abscissa in mm) the other direction is the cranio-caudal direction (CC) (ordinate in mm). A typical realisation is a network of 8×3 sensors (LM×CC) with a step of 37 mm. Its advantage is to leave smaller non-investigated volumes with a constant number of transducers.

In another example, the network is regular rectangular or square, with the main directions: the lateral-medial direction and the cranio-caudal direction. The step of the network is dictated by the number of sensors and by the size of the sensors. A typical realisation corresponds to a square network with a step of 45 mm with 9×3 transducers (LM×CC).

In another example, the network is non-regular, in such a way as to more densely cover the probable zones of positioning of the foetus or foetuses and of their heart. According to one example, one realisation involves densifying the triangular network under the bellybutton and above the bellybutton and moving apart the inter-transducer distances when moving away from the bellybutton in the direction LM.

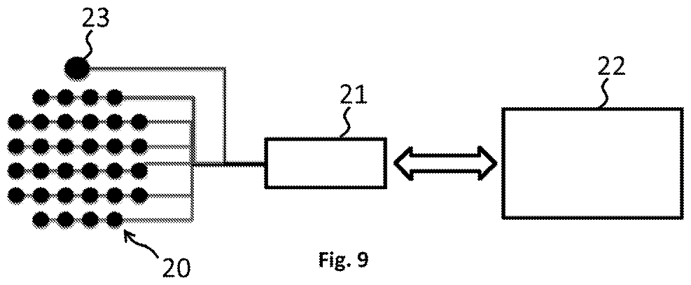

FIG. 9 shows a realisation of architecture of the system.

The control module 21 is linked or connected wirelessly to the processing module 22.

The electronic foetal surveillance system is composed of a collection of ultrasound sensors 20, a tocometer 23 in order to follow the uterine contractions.

In another example of realisation, other sensors complete or replace the tocometer 23, for example such as: a sensor of pulsed oxygen saturation (SpO2), an electrocardiogram, a thermometer, at least one microphone, an accelerometer, or an electromyogram.

The processing module 22 comprises electronics for control, for conditioning of the ultrasound signals and of the other signals coming from the other sensors, a unit for processing of the data, an element for storage of the data and an element for communication of the data.

In one realisation, the control module 21 and the processing module 22 are powered by a lithium-ion battery, or a lithium-polymer battery.

The processing module 22 for the digital processing of the data is either partly or completely integrated into the control module 21, or partly or completely integrated into a communicating terminal.

The communicating terminal is, for example:
A tablet computer,
A mobile telephone, in particular of the “smartphone” type,
A smartwatch,
A remote control,
A computer,
A smart television, or
A residential gateway.

This system allows to monitor the pregnancy remotely when the data is sent to a professional centre for control and remote assistance. Thus, this system forms a solution for remote surveillance of at-risk pregnancies at home or remote diagnosis in zones with a lack of medical care for which the patient is autonomous.

The electronics of the control module 21 are capable of controlling the X sensors (in one example, X=32) in order to carry out the conventional ultrasound modes. The control module 21 has Y active pathways (in one example, here Y=8), each active pathway has an emitter allowing to excite the ultrasound sensors, it is the information for control of the ultrasound waves. These emitters can be controlled independently (in phase, energy, amplitude, frequency and waveform) by the control module 21. In order to address the Y pathways of the system to the X sensors a multiplexing stage allows to address in real time a pathway Y to a sensor X in an arbitrary manner. The control module 21 accepts to control the Y pathways with complete independence. In order to protect the reception stages of the control module 21 switches Tx/Rx are positioned upstream of the reception pathways in order to protect the input stages of the control module 21. The reception stages of the control module 21 are composed of an impedance adapter, a linear amplifier, a variable-gain amplifier (for the compensation for the ultrasound attenuation of the medium), an analogue-filter stage and a stage for analogue-digital conversion (ADC).

The use of a plurality of ultrasound sensors allows to have as an advantage the possibility of exciting the transducers of the network according to sequences of different spatio-temporal ultrasound transmissions and receptions.

In one realisation of the control module, the emitter is a source of voltage creating a waveform having a crest-to-crest amplitude of approximately 5 to 30V, having a duration of 2 to 20 microseconds and having a central frequency of 1 to 4 MHz; this waveform is repeated at a period of 0.1 ms to 10 ms. This voltage excites Y sensors out of the X available sensors via multiplexing.

In one realisation of the module, the list of the Y excited sensors is changed at each firing. The change according to the time of this list constitutes the excitation sequence of the transducers.

In one realisation of the reception stage of the control module, the analogue-digital conversion stage is followed by a digital demodulation. This demodulated signal constitutes the complex ultrasound Doppler signal.

In one realisation of the reception stage, the Doppler signals coming from the Y sensors are sampled in Nz=1 to 20 points (corresponding to different depths) at a rate of repetition corresponding to the repetition of the firings in order to form Y*Nz (here 8 to 160) complex signals varying over time.

When the sequence chosen leads to a sensor being periodically visited at a time interval that is a multiple of the period of firings of the emitter, then the temporal dynamics of the Nz signals are closely linked to the movements of the tissue located in the beam of said sensor at the depth correspond to each of the Nz depths probed.

The following examples describe excitation sequences:
1. Verification of the Proper Connectivity of the System

A simple sequence of test ultrasound firings is acquired, for example such as by firing with the X ultrasound transducers one after the other. The data received is tested (for example such as its power) in order to verify the proper connectivity of the components of the system. If the connectivity of the components is not satisfactory, a message to the user informs of a possible problem in the operation of the device.
2. Detection of Good Contact Between the Sensors and the Skin

A sequence is repeated on each of the sensors. An indicator of quality of the signal is measured according to the sensor. This indicator allows to map the good quality of the signal and to detect whether a skin/sensor contract is defective.
3. Sequence of Seeking the Heart Through the Network of Sensors and in Terms of Depth

All the sensors are used in succession over several cardiac cycles, by using long, low-frequency ultrasound pulses and long listening times in order to allow a measurement, in terms of depth and spatialised, of the Doppler power. The Doppler signal coming from each point of an ultrasound beam corresponds to the demodulated signal back-scattered by this point, filtered over time by a high-pass filter, a typical cut-off frequency of which is 90 Hz. Its instantaneous average frequency is proportional to the axial particle speed of the tissue located at the position of interest. Its power (called Doppler) is linked to the number of mobile, reflective particles present near the point of interest.

The position of the foetal heart corresponds to the spatial position that sends back a Doppler power with the greatest powers, with the dimensions and the periodicity that fall within plausible intervals according to the foetal information. When the invention is used to monitor a multiple pregnancy, the positions of the N various foetuses (for N less than or equal to 8) are estimated by detecting the positions of the N most powerful Doppler signals back-scattered by the tissue with dimensions and periodicity that are plausible with the foetal information.
4.Optimisation and Feedback Loop for Belt Repositioning

The sequences 2 and 3 can be combined in order to give feedback information to the user in order to optimise the positioning.
5. Cardiac Monitoring with Sub-Populations of Sensors (the closest ones)

The sequence 3 can be used with a subgroup of sensors (typically the Y=7 or 8 sensors close to the initial position of the foetal heart) in order to monitor the heart for several minutes. In one realisation, these subgroups of sensors correspond to sensors neighbouring a central sensor that is included in the subgroup. The subgroup chosen first is that in which the central sensor is the sensor measures the maximum Doppler power. An advantage with respect to the simultaneous use of all the sensors (Y=X) is for example to reduce the electricity consumption or the exposure to the ultrasound waves.

In this configuration, the foetal cardiac rhythm is estimated by the following method on the basis of the Nz complex signals coming from the central sensor observed over a time of approximately 1 to 5 seconds.

First, these signals are filtered by a high-pass filter having a cut-off frequency of approximately 90 Hz. All the resulting signals or a portion of the resulting signals (Doppler signals) are then processed in such a way as to extract their instantaneous frequency according to the time. This instantaneous Doppler frequency is directly proportional to the projected speed in the direction of the beam of the particles present in the ultrasound beam relative to the central sensor and to its corresponding investigated depth.

The selection of the number of depths useful to the evaluation of axial speed of the tissue is, for example, carried out according to the average power of the Doppler signals.

When these particles are subjected to a periodic movement, such as that resulting from the beating of the foetal heart, then the instantaneous-frequency signal is periodic, and its period corresponds to the foetal cardiac period.

This thus involves estimating the period of the instantaneous-frequency signal by observing this signal over the 1 to 5 seconds of recording in order to extract therefrom its inverse: the FCR.

According to other examples, other approaches are selected to estimate the FCR:
A first approach is to take, as an estimator of the period of the signal, the non-zero positive value of the time that maximizes the autocorrelation function of the instantaneous Doppler frequency signal.
A second approach is to take, as an estimator of the FCR, the positive frequency value that maximizes the periodogram of the instantaneous Doppler frequency signal.
6. Verification of the Small Spatial Movements of the Heart

The sequence 5 can be used over several minutes in order to monitor the positioning of the heart and if necessary to optimise or restart the optimisation of the choice of the sensors of the subgroup of sensors of the network that fires.
7. Alternative Sequence: Heart Positioning/Foetal Movements/Amniotic Fluid Measurement

The sequence 3 is also used to map the cavities of amniotic fluid. The sequences 3 5 6 are alternated in order to optimise the monitoring of the foetal heart. One method of mapping of the cavities of amniotic fluid involves associating, with each position of each ultrasound beam of the X ultrasound transducers, the power information of the back-scattered signal. The amniotic fluid thus appears at the positions that send back a back-scattered ultrasound power that is very low with respect to the other surrounding tissue.

In one alternative, the sequence 3 is also used to map the foetal movements. One method of Doppler mapping of these movements involves first filtering, over time (that is to say according to the discrete sequence of successive firings), the demodulated ultrasound signals at each position of each beam through a band-pass filter (the lower cut-off frequency of which is approximately 30 Hz and the upper cut-off frequency of which is approximately 90 Hz), then calculating the average power of each of these signals at each point.
8. Determination of the Movements/Descent of the Foetus During Peripartum

The sequences 5 and 6 are alternated in order to monitor the movement of the foetus during its descent.
9. Mapping of the Movements and Travel

The sequence 3 is used to map the foetal movements and thus characterise foetal well-being.
10. Multi-Foetus (at least two foetuses)

The sequence 3 is used to detect a plurality of foetuses during a multiple pregnancy.
11. Doppler on Uterine Artery, Umbilical Cord

The sequence 3 is used to detect the maternal vessels or the umbilical cord and to measure the corresponding blood flow. It simultaneously allows the measurement of the maternal cardiac rhythm, and thus to distinguish foetal cardiac rhythm and maternal cardiac rhythm.

### NOMENCLATURE

20 ultrasound sensor
21control module
22 processing module
23 tocometer

1-10. (canceled)

11. An electronic system for foetal monitoring, comprising:
at least four ultrasound sensors to emit and receive ultrasound waves sequentially, the ultrasound sensors configured to be positioned on at least one maternal abdominal portion to cover a foetal cardiac structure in movement and perform measurements of a foetal cardiac frequency, a distance between two ultrasound sensors is maintained by a semi-rigid or semi-elastic link such that individual acoustic beams of the ultrasound sensors do not overlap, a length of the link is between 30 and 60 mm, a number of ultrasound sensors together form a triangular, square, rectangular or circular network;
a controller to control said at least four ultrasound sensors with a piece of control information to control the ultrasound waves sent to at least one ultrasound sensor;
a processor to process the ultrasound signals to continuously estimate a position of a foetal heart, a foetal cardiac rhythm and foetal movements based on the ultrasound signals received from said at least four ultrasound sensors.

12. The electronic system according to claim 11, wherein the length of the link is between 37 and 45 mm.

13. The electronic system according to claim 11, wherein the number of the ultrasound sensors is between 4 and 64.

14. The electronic system according to claim 11, wherein the number of ultrasound sensors is between 24 and 32.

15. The electronic system according to claim 11, wherein the piece of control information is a signal and one characteristic of the signal is predetermined by at least one of the elements chosen from: a phase, an energy, an amplitude, a frequency and a waveform.

16. The electronic system according to claim 15, wherein the piece of control information is independent from one ultrasound sensor to another and in real time from one ultrasound sensor to another.

17. The electronic system according to claim 11, wherein the processor is connected by a communication element to the electronic system, the communication element being wired or wireless.

18. The electronic system according to claim 11, wherein the link is a wireless link, the wireless link utilizes at least one of the following modes: radio waves, Ultra High Frequency (UHF), light waves, infrared light waves, sound waves, infrasound waves, ultrasound waves and one of the following network communication specifications: Bluetooth, Wi-Fi or ZigBee.

19. The electronic system according to claim 11, further comprising a communicating terminal configured to read or process data of the electronic system.

20. The electronic system according to claim 11, further comprising one of the elements chosen from: a tocodynamometer, a sensor of pulsed oxygen saturation (SpO2), an electrocardiogram, a thermometer, at least one microphone, an accelerometer, and an electromyogram.
